(19) United States
(12) Patent Application Publication
(10) Pub. No.: US 2012/0045401 A1
(43) Pub. Date: Feb. 23, 2012

(54) ORAL COMPOSITION

(75) Inventors: Shira Pilch, Highland Park, NJ (US); Malcolm Williams, Piscalaway, NJ (US)

(73) Assignee: Colgate-Palmolive Company, Piscataway, NJ (US)

(21) Appl. No.: 13/266,924

(22) PCT Filed: May 18, 2009
(86) PCT No.: PCT/US2010/044336

§ 371 Date: Oct. 28, 2011

(51) Int. Cl. A61K 8/81; C08F 222/06; A23G 4/06; A61Q 11/00; A61P 31/04; A61K 6/00; C08F 8/14

(52) U.S. Cl. 424/49; 523/120; 525/54.2; 525/54.26; 525/327.4; 525/327.5; 525/327.6; 525/327.7; 426/3

(57) ABSTRACT

The present invention provides an oral composition comprising a polymer, preferably a copolymer of methyl vinyl ether and maleic anhydride, attached to a sweetening agent via a cleavable linker, comprising a covalent bond, preferably an ester bond or an amide bond wherein the polymer is capable of attaching to a surface in an oral cavity, and the cleavable linker is cleavable in the oral cavity to release the sweetening agent.

### BACKGROUND OF THE INVENTION

Oral compositions, such as lozenges, chewing gum and mouthwash often comprise sweeteners to provide a sweet taste and improve breath odour. Once the composition is applied to the oral cavity a high level of the sweetener is quickly released providing an immediate taste sensation. However, this taste sensation quickly diminishes due to the adaptation of taste buds to the high level of sweetener and the removal of sweetener from the oral cavity in saliva to the stomach.

U.S. Pat. No. 6,464,961 and U.S. Pat. No. 6,315,987 disclose a polymer useful in oral care compositions which has a reactive group covalently bonded to a bactericide, flavorant and/or essential oil compound, said bond being hydrolysable in aqueous solution to slowly release said compound into said composition. U.S. Pat. No. 6,464,961 and U.S. Pat. No. 6,315,987 disclose the attachment of flavorants including menthol and thymol to polymers such as Gantrez®. However, neither U.S. Pat. No. 6,464,961 nor U.S. Pat. No. 6,315,987 disclose compositions comprising a sweetening agent, wherein the sweetening agent is slowly released into the composition, the sweetening agents impacting the taste via the interaction with taste buds on the tongue. Flavorants impact the olfactory response, they provide a smell via the interaction with olfactory neuron in the back of the nose. Additionally, sweetening agents often belong to different chemical classes from flavorants. As a result sweetening agents can exhibit different chemical and physical properties from flavorants. For example, most of the sweetening agents are water-soluble, while majority of the flavorants are oil soluble.

U.S. Patent Publication No. 2005/0260266 discloses an oral delivery system including at least one encapsulated active and a polymer matrix at least partially encapsulating the at least one active agent. The encapsulating material at least partially forms a physical barrier around the at least one active which prolongs the release of the active in the oral cavity in a controlled manner and/or enhances the uniformity of the release of the active in an oral cavity. The active component includes oral care actives, antibacterial agents, desensitizing agents, agents to counter breath odour, plaque acid buffering agents and sweeteners. The compositions comprise a carrier composition to accommodate the other components of the formulation. The carrier composition may comprise a film-forming polymer such as Gantrez™ as a means of reducing stain formation.

U.S. Pat. No. 5,661,221 discloses a process for preparing a cross-linked alkyl vinyl ether/maleic anhydride copolymer having improved linear viscoelastic properties desired in oral care preparations, wherein the cross-linking of an alkyl vinyl ether/maleic anhydride copolymer with a polyol compound is carried out at a temperature of from 50° to 90° and the cross-linked product is recovered. Suitable polyol cross-linking agents include sorbitol, xylitol, mannitol, sucrose, fructose, alkyl polyglucoside, glycerine, pentaerythrytol, ethylene glycol and polypropylene glycol. The cross-linking agent may be a sweetener such as sucrose. However, the sucrose would not be released in the oral cavity to act as a sweetening agent because it is a cross-linking agent. The polyols may be cleaved off via hydrolysis reaction. However, if the polyol is used as a cross-linking agent (at least two ester bonds connected to the polyol), it may take much longer for it to be hydrolyzed and released than that polyol is linked to the polymer via single ester bond.

Accordingly, it is an object of the present invention to overcome the disadvantages of known oral compositions comprising sweetening agents. It is an aim of the present invention to provide an improved oral composition which allows controlled release of the sweetening agent to thereby provide a longer taste sensation.

### BRIEF SUMMARY OF THE INVENTION

The present invention relates to an oral composition, a method for making an oral composition, a method for controlling the release of a sweetening agent in an oral composition, a method for enhancing and/or sustaining the sensation of flavour in an oral composition and the use of a polymer therefor.

In a first aspect the present invention provides an oral composition comprising a polymer attached to a sweetening agent via a cleavable linker, wherein the polymer is capable of attaching to a surface in the oral cavity, and the cleavable linker is cleavable in an oral cavity to release the sweetening agent.

The composition is advantageous because the sweetening agent is not immediately released into the oral cavity. The present compositions may allow prolonged and/or more uniform release of a sweetening agent compared to oral compositions comprising sweetening agents which are not attached to a polymer. Accordingly, the composition may enhance and/or sustain the sensation of flavour in an oral cavity. The composition may also provide a prolonged and/or uniform sweet taste and/or improvement of breath odour.

The polymer is also capable of attaching to a surface in the oral cavity. This is advantageous because the sweetening agent remains in the oral cavity for a longer period of time compared to oral compositions comprising sweetening agents which are not attached to a polymer.

The present invention also provides a portable dose article comprising the oral composition as defined above.

In a second aspect the present invention provides a method for making an oral composition as defined above, comprising attaching the polymer to the sweetening agent via the cleavable linker.

In a third aspect, the invention provides a method for controlling the release of a sweetening agent in an oral cavity, comprising applying to the oral cavity an oral composition as defined above or a portable dose article as defined above, wherein the cleavable linker is cleaved in the oral cavity to release the sweetening agent.

In a fourth aspect, the invention provides a use of a polymer for enhancing and/or sustaining the sensation of flavour in an oral cavity, wherein the polymer is attached to the sweetening agent via a cleavable linker, wherein the polymer is capable of attaching to a surface in the oral cavity and the cleavable linker is cleavable in an oral cavity to release the sweetening agent, so that the sensation of flavour is enhanced and/or sustained.

The present invention also provides a method for enhancing and/or sustaining the sensation of flavour in an oral cavity, comprising applying to the oral cavity an oral composition defined above or a portable dose article as defined above. It is reported (J. Agric. Food Chem. 1999, 47, 43364340) that there are cross-reactions between sweet taste and the perception of flavour. The perception of mint flavour drops significantly if the sweet taste disappears. If the sweet taste can prolonged, the potential to extend the perception of flavours is increased.

### DETAILED DESCRIPTION OF THE INVENTION

It should be understood that the detailed description and specific examples, while indicating embodiments of the invention, are intended for purposes of illustration only and are not intended to limit the scope of the invention.

The following definitions and non-limiting guidelines must be considered in reviewing the description of this invention set forth herein. The headings (such as “Introduction” and “Summary,”) and sub-headings (such as “Compositions” and “Methods”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof. Classification or discussion of a material within a section of this specification as having a particular utility (e.g., as being an “active” or a “carrier” ingredient) is made for convenience, and no inference should be drawn that the material must necessarily or solely function in accordance with its classification herein when it is used in any given composition.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations the stated of features. Specific Examples are provided for illustrative purposes of how to make and use the compositions and methods of this invention and, unless explicitly stated otherwise, are not intended to be a representation that given embodiments of this invention have, or have not, been made or tested.

As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

As referred to herein, all compositional percentages are by weight of the total composition, unless otherwise specified.

### Compositions

The present invention provides an oral composition comprising a sweetening agent attached to a polymer via a cleavable linker. The composition may be administered to or applied to a human or other animal subject.

The polymer is capable of attaching to a surface of the oral cavity. Typically the surface or layer which the polymer is capable of attaching to is selected from teeth, mucous membrane, gingival, cheek, tongue and lips. Preferably, the polymer is capable of attaching to all oral surfaces, including teeth, mucous membrane, gingival, cheek, tongue and lips. The polymer can adhere to the surface via charge to charge interaction or van der Waals interaction. In a preferred embodiment the polymer comprises a muco-adhesive polymer.

In a preferred embodiment the sweetening agent is released from the polymer at a rate which provides enhancement of flavour sensation and/or fresh breath in the oral cavity for a prolonged period of time compared to compositions comprising the same quantity of a sweetening agent which is not attached to a polymer. Preferably, the composition according to the present invention provides enhancement of flavour sensation in the oral cavity and/or fresh breath for at least twice, more preferably at least three times the period of time a composition comprising a sweetening agent which is not attached to a polymer provides a sweet taste in the oral cavity and/or fresh breath. Preferably the composition according to the present invention provides enhancement of flavour sensation in the oral cavity and/or fresh breath for at least five minutes.

The polymer in the oral composition according to the present invention is not particularly limited provided that it is capable of being attached to a sweetening agent via a cleavable linker and is suitable for administration to the oral cavity. Typically the polymer is a copolymer comprising at least one monomer suitable for attaching to the sweetening agent. Suitable monomers for attaching to the sweetening include acrylate, maleate and fumarate. The polymer may also comprise one or more different types of monomers including for example vinyl pyrrolidone, (meth)acrylic acid and vinyl acetate. In a preferred embodiment the polymer is a copolymer of maleic anhydride and methyl vinyl ether, such as Gantrez™.

In one embodiment, the polymer is not an alkyl vinyl ether/maleic anhydride copolymer cross-linked with a polyol compound, wherein the polyol compound is preferably sorbitol, xylitol, mannitol, sucrose, fructose, alkyl polyglucoside, glycerine, pentaerythrytol or ethylene glycol.

The cleavable linker which attaches the polymer to the sweetening agent may be any suitable linker capable of being cleaved in the oral cavity. In a preferred embodiment the linker is a covalent bond. In this embodiment the covalent bond may be formed from any suitable chemical reaction between the polymer and the sweetening agent. For example the linker may comprise an ester bond or an amide bond.

Each polymer may be attached to one compound of sweetening agent. Alternatively, each polymer may be attached to two or more of the same or different compounds of a sweetening agent.

The cleavable linker may be cleavable by any suitable means in the oral cavity. In a preferred embodiment the linker is cleaved by hydrolysis. The linker may be cleaved by an enzyme present in the oral cavity.

The sweetening agent in the composition according to the present invention is not particularly limited provided that it is capable of being attached to a polymer. Typically the sweetening agent is selected from dextrose, polydextrose, sucrose, maltose, dextrin, dried invert sugai, mannose, xylose, ribose, fructose, levulose, galactose, corn syrup, partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones, and mixtures thereof.

The amounts of the sweetening agent and the polymer is not particularly limited and may be any suitable amounts depending upon the required use of the sweetening agent. Typically the polymer is present in the composition in an amount of 0.001% to 10%. Typically the sweetening agent is present in the composition in an amount of from 0.005% to 5%, optionally from 0.01% to 1%.

The composition according to the present invention may also comprise one or more further agents typically selected from an anti-plaque agent, a whitening agent, antibacterial agent, cleaning agent, a flavouring agent, adhesion agents, surfactants, foam modulators, abrasives, pH modifying agents, humectants, mouth feel agents, colorants, abrasive, tartar control (anticalculus) agent, fluoride ion source, saliva stimulating agent, nutrient and combinations thereof.

In a preferred embodiment, the composition further comprises one or more flavouring agents. This embodiment is advantageous because the polymer linked sweetening agent is capable of enhancing and/or sustaining the sensation of flavour of the flavouring agent in an oral cavity.

Flavorants among those useful herein include any material or mixture of materials operable to improve the taste of the composition. Any orally acceptable natural or synthetic flavorant can be used, such as flavoring oils, flavoring aldehydes, esters, alcohols, similar materials, and combinations thereof. Flavorants include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavorants herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, [alpha]-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA), and mixtures thereof. One or more flavorants are optionally present in a total amount of 0.01% to 5%, optionally in various embodiments from 0.05 to 2%, from 0.1% to 2.5%, and from 0.1 to 0.5%.

Mouth-feel agents include materials imparting a desirable texture or other feeling during use of the composition.

Colorants among those useful herein include pigments, dyes, lakes and agents imparting a particular luster or reflectivity such as pearling agents. In various embodiments, colorants are operable to provide a white or light-colored coating on a dental surface, to act as an indicator of locations on a dental surface that have been effectively contacted by the composition, and/or to modify appearance, in particular color and/or opacity, of the composition to enhance attractiveness to the consumer. Any orally acceptable colorant can be used, including FD&C dyes and pigments, talc, mica, magnesium carbonate, calcium carbonate, magnesium silicate, magnesium aluminum silicate, silica, titanium dioxide, zinc oxide, red, yellow, brown and black iron oxides, ferric ammonium ferrocyanide, manganese violet, ultramarine, titaniated mica, bismuth oxychloride, and mixtures thereof. One or more colorants are optionally present in a total amount of 0.001% to 20%, for example 0.01% to 10% or 0.1% to 5%.

The compositions of the present invention further comprise an optional abrasive useful for example as a polishing agent. Any orally acceptable abrasive can be used, but type, fineness, (particle size) and amount of abrasive should be selected so that tooth enamel is not excessively abraded in normal use of the composition.

Suitable optional abrasives include silica, for example in the form of precipitated silica or as admixed with alumina, insoluble phosphates, calcium carbonate, and mixtures thereof. Among insoluble phosphates useful as abrasives are orthophosphates, polymetaphosphates and pyrophosphates. Illustrative examples are dicalcium orthophosphate dihydrate, calcium pyrophosphate, calcium pyrophosphate, tricalcium phosphate, calcium polymetaphosphate and insoluble sodium polymetaphosphate.

The compositions of the present invention optionally comprise a tartar control (anticalculus) agent. Tartar control agents among those useful herein include salts of any of these agents, for example their alkali metal and ammonium salts: phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-d iphosphonic acid), N-methyl azacyc lopentane-2,3-diphosphonic acid, ethane-1-hydroxy-1,1-diphosphonic acid (EH DP) and ethane-1-amino-1,1-diphosphonate, phosphonoalkane carboxylic acids and. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof.

The compositions of the present invention optionally comprise a fluoride ion source and useful, for example, as an anti-caries agent. Any orally acceptable particulated fluoride ion source can be used, including potassium, sodium and ammonium fluorides and monofluorophosphates, stannous fluoride, indium fluoride, amine fluorides such as olaflur (N′-octadecyltrimethylendiamine-N,N,N′-tris(2-ethanol)-dihydrofluoride), and mixtures thereof. One or more fluoride ion sources are optionally present in an amount providing a clinically efficacious amount of soluble fluoride ion to the oral composition.

The compositions of the present invention optionally comprise a saliva stimulating agent useful, for example, in amelioration of dry mouth. Any orally acceptable saliva stimulating agent can be used, including without limitation food acids such as citric, lactic, malic, succinic, ascorbic, adipic, fumaric and tartaric acids, and mixtures thereof. One or more saliva stimulating agents are optionally present in saliva stimulating effective total amount.

The compositions of the present invention optionally comprise a nutrient. Suitable nutrients include vitamins, minerals, amino acids, and mixtures thereof. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Nutritional supplements include amino acids (such as L-tryptophane, L-lysine, methionine, threonine, levocarnitine and L-carnitine), lipotropics (such as choline, inositol, betaine, and linoleic acid), and mixtures thereof.

In one embodiment, the oral composition according to the present invention is an oral care composition. As referred to herein, an “oral care composition” is any composition that is suitable for administration or application to the oral cavity of a human or animal subject for enhancing the health, hygiene or appearance of the subject, preferably providing such benefits as: the prevention or treatment of a condition or disorder of the teeth, gums, mucosa or other hard or soft tissue of the oral cavity; the prevention or treatment of a systemic condition or disorder; the provision of sensory, decorative or cosmetic benefits; and combinations thereof.

In various embodiments, the oral composition according to the present invention is not intentionally swallowed, but is rather retained in the oral cavity for a time sufficient to effect the intended utility. In other portable embodiments (such as a lozenge, mint, bead, wafer, liquid formulated for oral application from a small portable nebulizer, liquid formulated for oral application from a small portable drop-generating bottle, or a soft pliable tablet), the oral composition is intentionally swallowed, optionally after retention in the oral cavity for a time sufficient to effect intended utility.

The composition according to the present invention preferably comprises an orally acceptable carrier in a product such as mouthwash, toothpaste, dental cream, chewing gum, denture adhesive or portable dosage article such as, without limitation, a lozenge, a mint, bead, wafer, liquid formulated for oral application in a small portable nebulizer (spray bottle), liquid formulated for oral application in a small portable drop-generating bottle, or a soft pliable tablet (“chewie”). As used herein, an “orally acceptable carrier” refers to a material or combination of materials that are safe for use in the compositions of the present invention, commensurate with a reasonable benefit/risk ratio.

The present invention also provides portable dose article comprising an oral composition as defined above, wherein the portable dose article is selected from a lozenge, a mint, a bead, a wafer, a small portable nebulizer containing said composition in liquid formulated for oral application as a spray, a small portable bottle containing said composition in liquid formulated for oral application as a drop, and a soft pliable tablet.

Preferably, specific materials and compositions to be used in this invention are, accordingly, pharmaceutically- or cosmetically-acceptable, clinically effective, and/or clinically efficacious. As used herein, such a “pharmaceutically acceptable” or “cosmetically acceptable”, “clinically effective”, and/or “clinically efficacious” component is one that is suitable for use with humans and/or animals and is provided in an appropriate amount (a clinically efficacious amount) to provide the desired therapeutic, prophylactic, sensory, decorative, or cosmetic benefit without undue adverse side effects (such as toxicity, irritation, and allergic response) commensurate with a reasonable benefit/risk ratio.

The present invention also provides the use of a polymer for enhancing and/or sustaining the sensation of flavour in an oral cavity, wherein the polymer is attached to a sweetening agent via a cleavable linker, wherein the polymer is capable of attaching to a surface in the oral cavity and the cleavable linker is cleavable in the oral cavity to release the sweetening agent, so that the sensation of flavour is enhanced and/or sustained. The sweetening agent and polymer are preferably defined above in the first aspect of the present invention.

Methods of Manufacture. The present invention also provides a method for making an oral composition as defined above, comprising attaching the polymer to the sweetening agent via the cleavable linker.

As discussed above, the polymer may be attached to the sweetening agent by any suitable means, preferably by esterification.

The method may also comprise a step of mixing the polymer linked sweetening agent with an orally acceptable carrier to provide a formulation such as mouthwash, toothpaste, dental cream, or a lozenge (or into another portable foam embodiment as previously described herein to provide a portable dose article such as, without limitation, a lozenge, a mint, a bead, a wafer, a small portable nebulizer containing liquid formulated for oral application as a spray, a small portable bottle containing liquid formulated for oral application as a drop, or a soft pliable “chewie” tablet) through traditional processes for making these formulations as traditionally practiced by those of skill.

Methods of Use. The present invention also provides a method for controlling the release of a sweetening agent in an oral cavity, comprising applying to the oral cavity an oral composition as defined above or a portable dose article as defined above, wherein the cleavable linker is cleavable in the oral cavity to release the sweetening agent.

The present invention also provides a method for enhancing and/or sustaining the sensation of flavour in an oral cavity, comprising applying to the oral cavity an oral composition as defined above or a portable dose article as defined above.

The composition may be applied directly to teeth as dentifrice (such as, without limitation, toothpaste, or dental cream) or mouthwash. As referred to herein, “tooth” or “teeth” refers to natural teeth, dentures, dental plates, fillings, caps, crowns, bridges, dental implants, and the like, and any other hard surfaced dental prosthesis either permanently or temporarily fixed within the oral cavity. In this embodiment the dentifrice is agitated against each surface of the tooth with a toothbrush or the mouthwash is agitated against the teeth by vigorous swishing. As referred to herein, “applying” refers to any method by which the dentifrice or mouthwash is placed in contact with the tooth surface. Such methods, in various embodiments, comprise direct application of a composition by such methods as painting and brushing. In various embodiments, application of the composition comprises the use of an application device, which aids in maintaining contact of the composite to the tooth surface for sufficient time so as to allow cleaning.

The method according to the present invention may also include the steps of (a) ingesting into the oral cavity a safe and effective amount of formulated dosage of the composition as defined above from a portable dose article such as, without limitation, a lozenge, a mint, a bead, a wafer, a small portable nebulizer containing liquid formulated for oral application as a spray, a small portable bottle containing liquid formulated for oral application as a drop, or a soft pliable “chewie” tablet, (b) optionally retaining the dosage within the oral cavity for a period of time, and (c) swallowing the dosage.

In various embodiments, it is preferred that the subject does not eat or drink while the dentifrice and/or mouthwash oral composition is in contact with the dental surface. The oral care composition can be removed as and when required, at will, by an employment of standard oral hygiene procedures such as brushing or by rinsing, e.g., with water. The process can be repeated several times until the desired results, such as cleaning, are achieved.

In various embodiments, when the compositions of the present invention comprise one or more further active ingredients the compositions may be used for the treatment or prevention of disorders in the oral cavity, including cavity prevention, whitening, plaque prevention or reduction, gingivitis prevention or reduction, tartar control, sensitivity prevention or reduction, breath malodor prevention or reduction, and stain prevention. Compositions of the present invention may also be used for the treatment or prevention of systemic disorders, such as the improvement of overall systemic health characterized by a reduction in risk of development of systemic diseases, such as cardiovascular disease, stroke, diabetes, severe respiratory infection, premature and low birth weight infants (including associated post-partum dysfunction in neurologic/developmental function), and associated increased risk of mortality.

The present invention is further illustrated through the following non-limiting examples.

### Experimental Example 1: Toothpaste

| | |
|---|---|
| Cellulose gum | 0.650% |
| Polyethylene Glycol 600 | 3.000% |
| Sorbitol | 56.440% |
| Sodium Saccharin | 0.300% |
| Sodium Flouride | 0.243% |
| Tetrasodium Pyrophosphate-fine (FCC) | 0.500% |
| Polymer-Sweetener complex | 1.250% |
| Water | 9.967% |
| Colorant | 0.400% |
| Abrasive silica | 20.000% |
| Thickening silica | 4.250% |
| Flavor | 1.500% |
| Sodium Lauryl Sulfate | 1.500% |

### Experimental Example 2: Mouth Rinse

| | |
|---|---|
| Ethanol | 10% |
| Propylene Glycol | 7% |
| Sorbitol | 10% |
| Sodium Saccharin | 0.05% |
| Polymer-Sweetener complex | 0.3% |
| Water | 72% |
| Flavor | 0.25% |
| Sodium Lauryl Sulfate | 0.2% |
| Tauranol | 0.2% |

### Experimental Example 3: Film/Wafer

| | |
|---|---|
| Cellulose gum | 51% |
| Propylene Glycol | 8% |
| Tween 80 | 2 |
| Starch | 16% |
| Titanium Dioxide | 4% |
| Polymer-Sweetener complex | 10% |
| Saccharin | 0.3% |
| Flavor | 8.7% |

1. An oral composition comprising a polymer attached to a sweetening agent via a cleavable linker, wherein the polymer is capable of attaching to a surface in an oral cavity, and the cleavable linker is cleavable in the oral cavity to release the sweetening agent.

2. The oral composition according to claim 1, wherein the polymer is capable of attaching to the surface in the oral cavity selected from teeth, mucous membrane, gingival, cheek, tongue and lips.

3. The oral composition according to claim 1, wherein the polymer comprises a muco-adhesive polymer.

4. The oral composition according to claim 1, wherein the polymer is selected from a copolymer of maleic anhydride and methyl vinyl ether.

5. The oral composition according to claim 1, wherein the polymer is present in an amount of between 0.001% and 10%.

6. The oral composition according to claim 1, wherein the cleavable linker comprises a covalent bond.

7. The oral composition according to claim 6, wherein the linker comprises an ester bond or an amide bond.

8. The oral composition according to claim 1, wherein the cleavable linker is cleavable by hydrolysis.

9. The oral composition according to claim 1, wherein the cleavable linker is cleavable by an enzyme.

10. The oral composition according to claim 1, wherein the sweetening agent is selected from dextrose, polydextrose, sucrose, maltose, dextrin, dried invert sugar, mannose, xylose, ribose, fructose, levulose, galactose, corn syrup, partially hydrolyzed starch, hydrogenated starch hydrolysate, sorbitol, mannitol, xylitol, maltitol, isomalt, aspartame, neotame, saccharin and salts thereof, sucralose, dipeptide-based sweeteners, cyclamates, dihydrochalcones, and mixtures thereof.

11. The oral composition according to claim 1, wherein the sweetening agent is present in an amount from 0.005% to 5%.

12. The oral composition according to claim 1, wherein oral composition is adapted to release the sweetening agent from the polymer at a rate which provides enhancement of flavour sensation in the oral cavity for at least five minutes.

13. The oral composition according to claim 1, wherein the composition comprises an orally acceptable carrier for a toothpaste, a dental cream, a mouthwash, a chewing gum or a denture adhesive.

14. The oral composition according to claim 1, wherein the composition comprises one or more further agents selected from an anti-plaque agent, a whitening agent, antibacterial agent and a cleaning agent.

15. The oral composition according to claim 1, which further comprises a flavouring agent.

16. A portable dose article comprising the oral composition according to claim 1, wherein the portable dose article is selected from a lozenge, a mint, a bead, a wafer, a small portable nebulizer containing said composition in liquid formulated for oral application as a spray, a small portable bottle containing said composition in liquid formulated for oral application as a drop, and a soft pliable tablet.

17. A method for making the oral composition of claim 1, the method including attaching the polymer to the sweetening agent via the cleavable linker.

18. The method according to claim 17, wherein the polymer is attached to the sweetening agent by esterification.

19. A method for controlling the release of a sweetening agent in an oral cavity, comprising applying to the oral cavity the oral composition of claim 1.

20. Use of a polymer for enhancing and/or sustaining the sensation of flavour in an oral cavity, wherein the polymer is attached to a sweetening agent via a cleavable linker, wherein the polymer is capable of attaching to a surface in the oral cavity and the cleavable linker is cleavable in the oral cavity to release the sweetening agent, so that the sensation of flavour is enhanced and/or sustained.

21. Use according to claim 20, wherein the polymer is capable of attaching to the surface in the oral cavity selected from teeth, mucous membrane, gingival, cheek, tongue and lips.

22. Use according to claim 20, wherein the polymer is administered to the oral cavity in the oral composition comprising an orally acceptable carrier for a toothpaste, a dental cream, a mouthwash, a chewing gum or a denture adhesive.

23. A method for enhancing and/or sustaining the sensation of flavour in an oral cavity, comprising applying to the oral cavity the oral composition of claim 1.

24. A method for controlling the release of a sweetening agent in an oral cavity, comprising applying to the oral cavity the oral composition the portable dose article of claim 16, wherein the cleavable linker is cleaved in the oral cavity to release the sweetening agent.

25. A method for enhancing and/or sustaining the sensation of flavour in an oral cavity, comprising applying to the oral cavity the portable dose article of claim 16.
